(19) United States
(12) Patent Application Publication
(10) Pub. No.: US 2025/0215275 A1
(43) Pub. Date: Jul. 3, 2025

(54) Conductive Pressure Sensitive Adhesive

(71) Applicant: Henkel AG & Co. KGaA, Düsseldorf (DE)

(72) Inventors: Adrian Brandt, Essen (DE); Marlo Kropff, Düsseldorf (DE); Philipp Ernst, Solingen (DE)

(21) Appl. No.: 19/062,117

(22) Filed: Feb. 25, 2025

Related U.S. Application Data
(63) Continuation of application No. PCT/EP2023/073226, filed on Aug. 24, 2023.

(30) Foreign Application Priority Data
Aug. 25, 2022 (EP) 22192188.5

(51) Int. Cl. C09J 7/38; C09J 9/02; C09J 133/08

(52) U.S. Cl. CPC C09J 7/38; C09J 9/02; C09J 133/08; C09J 2301/302; C09J 2301/414; C09J 2301/50

(57) ABSTRACT

The present invention relates to a conductive pressure sensitive adhesive comprising a copolymer obtained from a reaction mixture comprising an acrylic monomer mixture and a special styrene derivative, a process for the production of said conductive pressure sensitive adhesive composition and its use. The present invention also relates to the acrylic monomer per se, processes for preparing it and uses thereof in polymerization processes.

The present invention relates to a conductive pressure sensitive adhesive comprising a copolymer obtained from a reaction mixture comprising an acrylic monomer mixture and a special styrene derivative, a process for the production of said conductive pressure sensitive adhesive composition and its use.

Conductive adhesives are primarily used for electronics, especially in technical fields such as aerospace and automotive. In recent years conductive adhesives have also become of interest for electrodes in medical and diagnostic applications such as electrocardiogra (ECG), electroencephalography (EEG) and electromyography (EMG).

In this regard US 2018/086948 provides an adhesive composition comprising a resin and an electro-conductive material, wherein the electro-conductive material is an ammonium salt of fluorosulfonic acid having 5 or more carbon atoms shown by the following general formula (1): (R¹—X—Z—SO₃⁻)ₙMⁿ⁺, wherein, R¹ represents a linear, branched, or cyclic monovalent hydrocarbon group having 1 to 40 carbon atoms and optionally substituted by a heteroatom or optionally interposed by a heteroatom; X represents any of a single bond, an ether group, an ester group, and an amide group; Z represents a linear or branched alkylene group having 2 to 4 carbon atoms, containing 1 to 6 fluorine atoms, and optionally containing a carbonyl group; Mⁿ⁺ represents a cation having one or two ammonium cation structures; and “n” is 1 when the number of the ammonium cation structure contained in the Mⁿ⁺ is one, or is 2 when the number of the ammonium cation structure contained in the Mⁿ⁺ is two.

WO 2020/178218 discloses an ionically conductive pressure sensitive adhesive composition comprising a) a (meth)acrylate resin comprising at least 10% of a (meth)acrylate monomer comprising OH-group by weight of the total weight of the (meth)acrylate resin; and b) an ionic liquid.

Despite the efforts already made, there is still the need for conductive pressure sensitive adhesive which can be applied to detect bio-signals such as heart rate or brain waves, especially in long-term monitoring. Another aspect is the problem of sustainability as the materials can usually only used once.

In order to address said needs, the present invention provides a conductive pressure sensitive adhesive based on materials obtainable from renewal sources. In this regard, a first aspect of the present invention is a conductive pressure sensitive adhesive composition comprising a copolymer obtained from a reaction mixture comprising
a) a monomeric acrylic mixture; and
b) a compound of Formula (I):

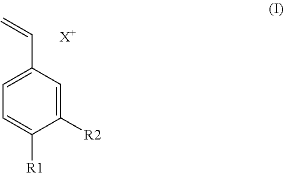

wherein R¹ is either a sulfate group (OSO₃⁻), a sulfonate group (SO₃⁻), or a hydrogen atom (H); R² is a sulfate group, a sulfonate group, an alkoxy group or a hydrogen atom,
and wherein X⁺ is a counter ion with the proviso that either R¹ or R² is a sulfate group (OSO₃⁻).

Within the course of the present invention, it was found that the conductive pressure sensitive adhesive composition of the present invention provides improved ion conductivity and increased peel strength, even in the absence of an ionic liquid as commonly employed. The inventive composition further has the advantage that the compound of Formula (I) can be incorporated into the acrylate polymeric backbone.

The conductive pressure sensitive adhesive composition according to the present invention can advantageously be used as a dry film, which offers a solution for long-term monitoring of biosignals by acting as a functional contact between electrode and skin. In contrast to gel-type electrodes currently in the market it does not have the draw-back of drying out or causing skin irritation. Furthermore, the impedance of the conductive pressure sensitive adhesive composition according to the present invention is very low without any addition of water.

The counter ion X⁺ is preferably selected from the group of:
NR³R⁴R⁵R⁶⁺, with each of R³, R⁴, R⁵ and R⁶ being independently selected from a C₁-C₈-alkyl,
an optionally substituted imidazolium,
choline,
an optionally substituted pyridinium,
an optionally substituted pyrrolidinium,
phosphonium,
sulfonium,
and mixtures thereof.

The counter ion X⁺ may not be a mixture of the cations, but a single species, preferably selected from one of those defined above.

Where X⁺ is NR³R⁴R⁵R⁶⁺ as defined above, each of R³, R⁴, R⁵ and R⁶ is independently selected from a C₂-C₆-alkyl. Optionally, R³, R⁴, R⁵ and R⁶ are all the same alkyl moiety. Preferably X⁺ is N(C₄H₉)₄_{⁺}.

Where X⁺ is an optionally substituted imidazolium, options for X⁺ include unsubstituted imidazolium or a dialkylimidazolium. Preferably X⁺ is a 1,3-dialkylimidazolium, with each alkyl group being independently selected from a linear or branched C1-C6 alkyl, more preferably a linear or branched C1-C3 alkyl. Preferably, each alkyl group is independently selected from a linear C1-C6 alkyl, more preferably a linear C1-C3 alkyl. A particularly suitable group is 1-ethyl-3-methylimidazolium. X⁺ may also be tris(2-hydroxyethyl)methylammonium or 1-allyl-3-methylimidazolium.

Where X⁺ is an optionally substituted pyridinium, options for X⁺ include unsubstituted pyridinium or a singly-N-substituted pyridinium, for example a 1-alkylpyridinium, with the alkyl group being selected from a linear or branched C1-C6 alkyl, preferably a linear or branched C1-C4 alkyl, more preferably a linear C1-C4 alkyl. X⁺ may also include a doubly-substituted pyridinium, i.e. with substituents at the 1,2 or 1,3, or 1,4 positions on the pyridine ring. For example a 1,2-alkylpyridinium, 1,3-alkylpyridinium or a 1,4-alkylpyridinium, with each alkyl group being independently selected from a linear or branched C1-C6 alkyl, preferably a linear or branched C1-C4 alkyl, more preferably a linear C1-C4 alkyl.

Where X⁺ is an optionally substituted pyrrolidinium, options for X⁺ include unsubstituted pyrrolidinium or a singly-N-substituted pyrrolidinium, for example a 1-alkylpyrrolidinium, with the alkyl group being selected from a linear or branched C1-C6 alkyl, preferably a linear or branched C1-C4 alkyl, more preferably a linear C1-C4 alkyl. X⁺ may also include a doubly-substituted pyrrolidinium. For example, the nitrogen ring member may be doubly-substituted. For example, X⁺ may be a 1,1-dialkyl pyrrolidinium, with each alkyl group being independently selected from a linear or branched C1-C6 alkyl, preferably a linear or branched C1-C4 alkyl, more preferably a linear C1-C4 alkyl.

With respect to the groups R1 and R2, R1 is preferably a sulfate group and R2 is an alkoxy group or a hydrogen atom. In an especially preferred embodiment, R1 is a sulfate group and R2 is a hydrogen atom.

The alkoxy group of the compound of Formula (I), if present, is preferably selected from the group of OMe, OEt, OPr and OBu, with preference given to OMe.

In an especially preferred embodiment, the compound of Formula (I) is the following:

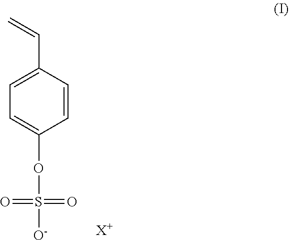

with X⁺ defined as above. Preferably, X⁺ is N(C₄H₉)₄_{⁺}.

The conductive pressure sensitive adhesive comprises a copolymer obtained from a reaction mixture comprising a monomeric acrylic mixture. In a preferred embodiment, the monomeric acrylic mixture comprises at least one of the following: methyl methacrylate, hydroxyethyl acrylate, hydroxypropyl acrylate, hydroxybutyl acrylate, n-butyl acrylate, 2-ethylhexyl acrylate, acrylic acid, C1-C18 alkyl (meth)acrylate, (meth)acrylamide, vinyl acetate, N-vinyl caprolactame, acrylonitrile, vinyl ether, benzyl (meth)acrylate, cyclohexyl (meth)acrylate, glycidyl (meth)acrylate and mixtures thereof, preferably formed from the monomers selected from the group consisting of hydroxyethyl acrylate, methyl methacrylate, n-butyl acrylate, 2-ethylhexyl acrylate and mixtures thereof.

Especially preferred is an embodiment of the present invention wherein the monomeric acrylic mixture comprises at least one, preferably all, of the following 2-ethylhexyl acrylate (abbreviation: 2-EHA), methyl methacrylate (abbreviation: MMA), 2-hydroxyethyl acrylate (abbreviation: 2-HEA), n-butyl acrylate (abbreviation: BA).

In a preferred embodiment of the conductive pressure sensitive adhesive composition according to the invention, the reaction mixture contains 2-EHA in an amount of 1 to 20 wt.-%, preferably 2 to 7 wt.-% in the reaction mixture, based on the total weight of the reaction mixture.

In a preferred embodiment of the conductive pressure sensitive adhesive composition according to the invention, the reaction mixture contains MMA in an amount of 1 to 7 wt.-%, preferably 2 to 5 wt.-% in the reaction mixture, based on the total weight of the reaction mixture.

In a preferred embodiment of the conductive pressure sensitive adhesive composition according to the invention, the reaction mixture contains 2-HEA in an amount of 15 to 40 wt.-%, preferably 20 to 35 wt.-% in the reaction mixture, based on the total weight of the reaction mixture.

In a preferred embodiment of the conductive pressure sensitive adhesive composition according to the invention, the the reaction mixture contains BA in an amount of 40 to 80 wt.-%, preferably 55 to 65 wt.-% in the reaction mixture, based on the total weight of the reaction mixture.

In a preferred embodiment of the conductive pressure sensitive adhesive composition according to the invention, the compound of formula (I) is contained in an amount of 0.1 to 50 wt.-%, preferably 0.2 to 30 wt.-%, especially 0.5 to 10 wt.-%, in the reaction mixture, based on the total weight of the reaction mixture.

Within the course of the present invention, it was surprisingly found that addition of an ionic liquid to the conductive pressure sensitive adhesive composition enables faster build-up of peel strength over time compared with compositions merely comprising either an ionic liquid or the compound of Formula (I). Therefore, in a preferred embodiment, the conductive pressure sensitive adhesive composition further comprises an ionic liquid.

The ionic liquid optionally comprised in conductive pressure sensitive adhesive composition is preferably a non-toxic, non-irritating ionic liquid leading to ionic conductivity.

More specifically, the ionic liquid is selected from the group consisting of imidazolium acetates, imidazolium sulfonates, imidazolium chlorides, imidazolium sulphates, imidazolium phosphates, imidazolium thiocyanates, imidazolium dicyanamides, imidazolium benzoates, imidazolium triflates, choline triflates, choline saccharinate, choline sulfamates, pyridinium acetates, pyridinium sulfonates, pyridinium chlorides, pyridinium sulphates, pyridinium phosphates, pyridinium thiocyanates, pyridinium dicyanamides, pyridinium benzoates, pyridinium triflates, pyrrolidinium acetates, pyrrolidinium sulfonates, pyrrolidinium chlorides, pyrrolidinium sulphates, pyrrolidinium phosphates, pyrrolidinium thiocyanates, pyrrolidinium dicyanamides, pyrrolidinium benzoates, pyrrolidinium triflates, phosphonium acetates, phosphonium sulfonates, phosphonium chlorides, phosphonium sulphates, phosphonium phosphates, phosphonium thiocyanates, phosphonium dicyanamides, phosphonium benzoates, phosphonium triflates, sulfonium acetates, sulfonium sulfonates, sulfonium chlorides, sulfonium sulphates, sulfonium phosphates, sulfonium thiocyanates, sulfonium dicyanamides, sulfonium benzoates, sulfonium triflates, ammonium acetates, ammonium sulfonates, ammonium chlorides, ammonium sulphates, ammonium phosphates, ammonium thiocyanates, ammonium dicyanamides, ammonium benzoates, ammonium triflates and mixtures thereof.

Preferably, said ionic liquid is selected from the group consisting of 1-ethyl-3-methylimidazolium acetate, 1-ethyl-3-methylimidazolium methane sulfonate, 1-ethyl-3-methylimidazolium trifluoromethane sulfonate, 1-ethyl-3-methylimidazolium chloride, 1-ethyl-3-methylimidazolium ethyl sulphate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, choline trifluoromethanesulfonate, choline saccharinate, choline acesulfamate, choline N-cyclohexylsulfamate, tris(2-hydroxyethyl)methylammonium methyl sulphate, 1-ethyl-3-methylimidazolium tetrafluoroborate, 1-allyl-3-methylimidazolium bis(trifluoromethylsulfonyl)imide, choline acetate and mixtures thereof.

More preferably, the ionic liquid is selected from the group consisting of 1-ethyl-3-methylimidazolium benzoate, 1-ethyl-3-methylimidazolium tetrafluoroborate, 1-ethyl-3-methylimidazolium methane sulfonate, 1-ethyl-3-methylimidazolium chloride, 1-ethyl-3-methylimidazolium trifluoromethane sulfonate, choline trifluoromethane sulfonate, 1-ethyl-3-methylimidazolium acetate, choline acetate, 1-ethyl-3-methylimidazolium diethylphosphate, 1-allyl-3-methylimidazolium bis(trifluoromethylsulfonyl)imide, 1-ethyl-3-methylimidazolium ethyl sulphate, 1-ethyl-3-methylimidazolium thiocyanate, 1-ethyl-3-methylimidazolium dicyanamide, choline saccharinate, choline acesulfamate, and mixture thereof.

In one embodiment of the conductive pressure sensitive adhesive composition, two or more ionic liquids are used, in this embodiment said ionic liquids are selected from the group consisting of 1-ethyl-3-methylimidazolium acetate, 1-ethyl-3-methylimidazolium methane sulfonate, 1-ethyl-3-methylimidazolium trifluoromethane sulfonate, 1-ethyl-3-methylimidazolium chloride, 1-ethyl-3-methylimidazolium ethyl sulphate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, choline trifluoromethanesulfonate, choline saccharinate, choline acesulfamate, choline N-cyclohexylsulfamate, tris(2-hydroxyethyl)methylammonium methyl sulphate, 1-ethyl-3-methylimidazolium tetrafluoroborate, 1-allyl-3-methylimidazolium bis(trifluoromethylsulfonyl)imide, choline acetate; preferably two or more ionic liquids are selected from the group consisting of 1-ethyl-3-methylimidazolium benzoate, 1-ethyl-3-methylimidazolium tetrafluoroborate, 1-ethyl-3-methylimidazolium methane sulfonate, 1-ethyl-3-methylimidazolium chloride, 1-ethyl-3-methylimidazolium trifluoromethane sulfonate, choline trifluoromethane sulfonate, 1-ethyl-3-methylimidazolium acetate, choline acetate, 1-ethyl-3-methylimidazolium diethylphosphate, 1-allyl-3-methylimidazolium bis(trifluoromethylsulfonyl)imide, 1-ethyl-3-methylimidazolium ethyl sulphate, 1-ethyl-3-methylimidazolium thiocyanate, 1-ethyl-3-methylimidazolium dicyanamide, choline saccharinate, choline acesulfamate.

In an especially preferred embodiment, the ionic liquid is selected from the group consisting of 1-ethyl-3-methylimidazolium trifluoromethanesulfonate, 1-ethyl-3-methylimidazolium acetate, 1-ethyl-3-methylimidazolium methanesulfonate, 1-ethyl-3-methylimidazolium chloride, 1-ethyl-3-methylimidazolium sulfate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, 1-ethyl-3-methylimidazolium tetrafluoroborate, choline trifluoromethanesulfonate, choline saccharinate, choline acesulfamate, choline N-cyclohexylsulfamate, tris(2.hydroxyethyl)methylammonium methyl sulfate, choline acetate and mixtures thereof, and more preferably selected from the group consisting of 1-ethyl-3-methylimidazolium trifluoromethanesulfonate, 1-ethyl-3-methylimidazolium acetate, 1-ethyl-3-methylimidazolium methanesulfonate, 1-ethyl-3-methylimidazolium chloride, 1-ethyl-3-methylimidazolium sulfate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, 1-ethyl-3-methylimidazolium tetrafluoroborate, choline trifluoromethanesulfonate, choline saccharinate, choline acesulfamate, choline acetate, and mixtures thereof.

Suitable commercially available ionic liquids for use in the present invention include, but are not limited to Basionics ST80, Basionics Kat1, Basionics BC01, Basionics VS11, Basionics VS03, and Efka IO 6785, all from BASF.

The ionic liquid is preferably comprised in the conductive pressure sensitive adhesive composition in an amount of from 0.1 to 35% by weight of the total weight of the composition, preferably from 0.5 to 25%, and more preferably from 1 to 15%.

Suitably, the conductive pressure sensitive adhesive composition comprises a copolymer obtained from a reaction mixture comprising:
a. 2-EHA
b. MMA
c. 2-HEA
d. BA
e. compound of Formula (I); and an ionic liquid.

It was surprisingly found that the best performance of the conductive pressure sensitive adhesive composition was achieved if a mixture of acrylic monomers was used to obtain the copolymer. Therefore, in a preferred embodiment, the conductive pressure sensitive adhesive composition the composition comprises a copolymer obtained from a reaction mixture comprising:
a. 2-EHA in an amount of 1 to 20 wt.-%, preferably 2 to 7 wt.-%
b. MMA in an amount of 1 to 7 wt.-%, preferably 2 to 5 wt.-%
c. 2-HEA in an amount of 15 to 40 wt.-%, preferably 20 to 35 wt.-%
d. BA in an amount of 40 to 80 wt.-%, preferably 55 to 65 wt.-%
e. compound of Formula (I) in an amount of 0.1 to 50 wt.-%, preferably 0.2 to 30 wt.-%, especially 0.5 to 10 wt.-%,
   based on the total weight of the reaction mixture, respectively; and an ionic liquid in the amount of from 0.1 to 35 wt.-%, preferably from 0.5 to 25 wt.-%, and more preferably from 1 to 15 wt.-%, based on the total weight of the conductive pressure sensitive adhesive composition, respectively.

In another aspect, the present invention refers to a process for the production of the conductive pressure sensitive adhesive composition according to the invention, the process comprising the following steps:
a) providing an initial and a feed solution of a reaction mixture comprising a monomeric acrylic mixture and a compound of formula (I);
b) providing an initial and a feed solution of an initiator solution;
c) adding the initial initiator solution to the initial solution of the reaction mixture at an ambient temperature;
d) heating the reaction mixture of step c) to temperatures between 60° C. and 120° C., preferably to temperatures between 70° C. and 90° C.;
e) continuously adding the feed solution of the reaction mixture and feed solution of the initiator solution.

In a preferred embodiment, step e) of the inventive process is carried out over a period of 2 to 8 hours, preferably over 3 to 5 hours.

The initiator employed may be any suitable component for initiating the polymerization of acrylic and styrenic monomers. Preferably, the initiator is selected from the group consisting of 2,2′-azobisisobutyronitril, 1,1′-Azobis(cyclohexanecarbonitrile), Dimethyl 2,2′-azobis(2-methylpropionate), 2,2′-Azobis(2,4-dimethylvaleronitrile),2,2′-Azobis(2-methylbutyronitrile), Benzoyl peroxide, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide. Special preference is given to 2,2′-azobisisobutyronitril (AIBN) as initiator.

The conductive pressure sensitive adhesive composition of the present invention is particularly suitable to be brought into contact with human skin and for employment in long-term monitoring of body functions such as heart rate or brain waves. Therefore, a further object of the present invention is the use of the conductive pressure sensitive adhesive composition according to the invention in skin applications as a contact medium as part of electrodes measuring bio-signals from the skin, athletic tape, transdermal drug patches, permanent adhesive tapes for industrial use and permanent adhesive tapes for construction work. In addition, the conductive pressure sensitive adhesive is preferably used in applications for safety labels for power equipment, masking tapes, price marking labels and/or electrochemical debonding.

The present invention also relates to a compound of Formula (I):

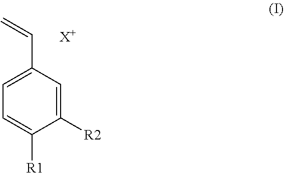

wherein R1, R2 and X⁺ are as defined herein.

Both R1 and R2 may be a sulfate group. Alternatively, one of R1 or R2 is a sulfate group. Preferably, R1 is a sulfate group and R2 is a hydrogen atom.

The present invention also provides a process for preparing a compound of formula (I) as defined herein. The process broadly involves preparing the free acid that corresponds to the compound of formula (I) an alternative salt thereof, followed by cation exchange with a salt based on the appropriate cation (i.e. a base where X⁺ is the compound (I) cation) to yield the salt of compound (I). Thus, the process may also involve preparing the free acid form of compound (I), and addition of a salt based on the appropriate cation (i.e. a base where X⁺ is the desired cation) to yield the salt of compound (I). The process may also involve preparing an alternative salt of compound (I), i.e. where X⁺ is different to the X⁺ that is desired, and performing cation exchange with a salt based on the appropriate cation (i.e. a base where X⁺ is the desired cation) to yield the salt of compound (I).

An exemplary process for preparing a compound of formula (IA) comprises:
reacting 4-vinylphenol and sulfur trioxide trimethylamine complex to form a reaction mixture,
adding tetrabutylammonium hydroxide to the reaction mixture,
and isolating the compound of formula (IA)

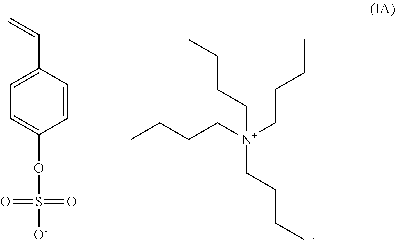 [SMILES: C=Cc1ccc(OS(=O)(=O)[O-])cc1 ; CCCC[N+](CCCC)(CCCC)CCCC]

As will be well understood by the skilled person, the starting materials and reagents can be appropriately replaced in order to prepare other compounds falling within the scope of the compound of formula (I).

The present invention also relates the use of a compound of Formula (I):

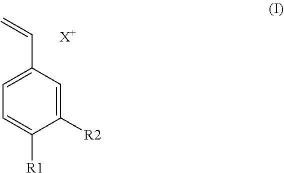

wherein R1, R2 and X⁺ are as defined herein as a monomer in a polymerization process.

It will be understood by the current disclosure that the invention may also be described by the following statements:

1. Conductive pressure sensitive adhesive composition comprising a copolymer obtained from a reaction mixture comprising
a) a monomeric acrylic mixture; and
b) a compound of Formula (I):

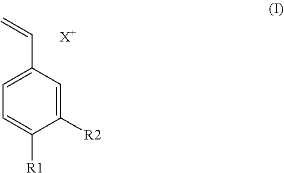

wherein R¹ is either a sulfate group (OSO₃⁻), a sulfonate group (SO₃⁻), or a hydrogen atom (H); R² is a sulfate group, a sulfonate group, an alkoxy group or a hydrogen atom, and wherein X⁺ is a counter ion with the proviso that either R¹ or R² is a sulfate group.
2. Conductive pressure sensitive adhesive composition according to statement 1, characterized in that R1 is a sulfate group (OSO₃⁻).
3. Conductive pressure sensitive adhesive composition according to statement 2, characterized in that R2 is a hydrogen atom.
4. Conductive pressure sensitive adhesive composition according to statement 2, characterized in that R2 is an alkoxy group.
5. Conductive pressure sensitive adhesive composition according to statement 1, characterized in that R2 is a sulfate group (OSO₃⁻).
6. Conductive pressure sensitive adhesive composition according to statement 5, characterized in that R1 is a hydrogen atom.
7. Conductive pressure sensitive adhesive composition according to statement 5, characterized in that R1 is an alkoxy group.
8. Conductive pressure sensitive adhesive composition according to any of the forgoing statements, characterized in that X⁺ is selected from the group of NR³R⁴R⁵R⁶⁺, with each of R³, R⁴, R⁵ and R⁶ being independently selected from C₁-C₈-alkyl, preferably C₂-C₆-alkyl, more preferably X⁺ is N(C₄H₉)₄_{⁺}.
9. Conductive pressure sensitive adhesive composition according to any of the forgoing statements, characterized in that X⁺ is an optionally substituted imidazolium.
10. Conductive pressure sensitive adhesive composition according to statement 9, characterized in that X⁺ is imidazolium.
11. Conductive pressure sensitive adhesive composition according to statement 9, characterized in that X⁺ is a substituted imidazolium, preferably a 1,3-dialkylimidazolium, with each alkyl group being independently selected from a linear or branched C1-C6 alkyl, more preferably a linear or branched C1-C3 alkyl.
12. Conductive pressure sensitive adhesive composition according to statement 9, characterized in that X⁺ is a 1,3-dialkylimidazolium, with each alkyl group being independently selected from a linear C1-C6 alkyl, preferably a linear C1-C3 alkyl.
13. Conductive pressure sensitive adhesive composition according to statement 9, characterized in that X⁺ is 1-ethyl-3-methylimidazolium.
14. Conductive pressure sensitive adhesive composition according to any one of statements 1 to 7, characterized in that X⁺ is choline.
15. Conductive pressure sensitive adhesive composition according to any one of statements 1 to 7, characterized in that X⁺ is an optionally substituted pyridinium.
16. Conductive pressure sensitive adhesive composition according to statement 15, characterized in that X⁺ is pyridinium.
17. Conductive pressure sensitive adhesive composition according to statement 15, characterized in that X⁺ is a substituted pyridinium, preferably a singly-N-substituted pyridinium or a doubly-substituted pyridinium, i.e. with substituents at the 1,2 or 1,3, or 1,4 positions on the pyridine ring, more preferably X⁺ is a 1-alkylpyridinium, a 1,2-alkylpyridinium, 1,3-alkylpyridinium or a 1,4-alkylpyridinium, with each alkyl group being independently selected from a linear or branched C1-C6 alkyl, preferably a linear or branched C1-C4 alkyl, more preferably a linear C1-C4 alkyl.
18. Conductive pressure sensitive adhesive composition according to any one of statements 1 to 7, characterized in that X⁺ is an optionally substituted pyrrolidinium.
19. Conductive pressure sensitive adhesive composition according to statement 18, characterized in that X⁺ is pyrrolidinium.
20. Conductive pressure sensitive adhesive composition according to statement 18, characterized in that X⁺ is a substituted pyrrolidinium, preferably a singly-N-substituted pyrrolidinium or a doubly-substituted pyrrolidinium, for example, the nitrogen ring member being doubly-substituted, more preferably a 1-alkylpyrrolidinium or a 1,1-dialkyl pyrrolidinium, with each alkyl group being independently selected from a linear or branched C1-C6 alkyl, preferably a linear or branched C1-C4 alkyl, more preferably a linear C1-C4 alkyl.
21. Conductive pressure sensitive adhesive composition according to any one of statements 1 to 7, characterized in that X⁺ is phosphonium.
22. Conductive pressure sensitive adhesive composition according to any one of statements 1 to 7, characterized in that X⁺ is sulfonium.
23. Conductive pressure sensitive adhesive composition according to any of the forgoing statements, characterized in that the (meth)acrylic mixture comprises at least one of the following: methyl methacrylate, hydroxyethyl acrylate, hydroxypropyl acrylate, hydroxybutyl acrylate, n-butyl acrylate, 2-ethylhexyl acrylate, acrylic acid, C1-C18 alkyl (meth)acrylate, (meth)acrylamide, vinyl acetate, N-vinyl caprolactame, acrylonitrile, vinyl ether, benzyl (meth)acrylate, cyclohexyl (meth)acrylate, glycidyl (meth)acrylate and mixtures thereof, preferably formed from the monomers selected from the group consisting of hydroxyethyl acrylate, methyl methacrylate, n-butyl acrylate, 2-ethylhexyl acrylate and mixtures thereof.
24. Conductive pressure sensitive adhesive composition according to any of the forgoing statements, characterized in that the compound of formula (I) is contained in an amount of 0.1 to 50 wt.-%, in the reaction mixture, based on the total weight of the reaction mixture.
25. Conductive pressure sensitive adhesive composition according to any of the forgoing statements, characterized in that the compound of formula (I) is contained in an amount of 0.2 to 30 wt.-%, in the reaction mixture, based on the total weight of the reaction mixture.
26. Conductive pressure sensitive adhesive composition according to any of the forgoing statements, characterized in that the compound of formula (I) is contained in an amount of 0.5 to 10 wt.-%, in the reaction mixture, based on the total weight of the reaction mixture.
27. Conductive pressure sensitive adhesive composition according to any of the forgoing statements, characterized in that reaction mixture contains 2-EHA in an amount of 1 to 10 wt.-%, in the reaction mixture, based on the total weight of the reaction mixture.
28. Conductive pressure sensitive adhesive composition according to any of the forgoing statements, characterized in that reaction mixture contains 2-EHA in an amount of 2 to 7 wt.-% in the reaction mixture, based on the total weight of the reaction mixture.
29. Conductive pressure sensitive adhesive composition according to any of the forgoing statements, characterized in that the reaction mixture contains MMA in an amount of 1 to 7 wt.-%, in the reaction mixture, based on the total weight of the reaction mixture.
30. Conductive pressure sensitive adhesive composition according to any of the forgoing statements, characterized in that the reaction mixture contains MMA in an amount of 2 to 5 wt.-% in the reaction mixture, based on the total weight of the reaction mixture.
31. Conductive pressure sensitive adhesive composition according to any of the forgoing statements, characterized in that the reaction mixture contains 2-HEA in an amount of 15 to 40 wt.-%, in the reaction mixture, based on the total weight of the reaction mixture.
32. Conductive pressure sensitive adhesive composition according to any of the forgoing statements, characterized in that the reaction mixture contains 2-HEA in an amount of 20 to 35 wt.-% in the reaction mixture, based on the total weight of the reaction mixture.
33. Conductive pressure sensitive adhesive composition according to any of the forgoing statements, characterized in that the reaction mixture contains BA in an amount of 40 to 80 wt.-%, in the reaction mixture, based on the total weight of the reaction mixture.
34. Conductive pressure sensitive adhesive composition according to any of the forgoing statements, characterized in that the reaction mixture contains BA in an amount of 55 to 65 wt.-% in the reaction mixture, based on the total weight of the reaction mixture.
35. Conductive pressure sensitive adhesive composition according to any of the forgoing statements, characterized in that the pressure sensitive adhesive composition further comprises an ionic liquid, preferably selected from the group consisting of 1-ethyl-3-methylimidazolium trifluoromethanesulfonate, 1-ethyl-3-methylimidazolium acetate, 1-ethyl-3-methylimidazolium methanesulfonate, 1-ethyl-3-methylimidazolium chloride, 1-ethyl-3-methylimidazolium sulfate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, 1-ethyl-3-methylimidazolium tetrafluoroborate, choline trifluoromethanesulfonate, choline saccharinate, choline acesulfamate, choline N-cyclohexylsulfamate, tris(2.hydroxyethyl)methylammonium methyl sulfate, choline acetate and mixtures thereof, and more preferably selected from the group consisting of 1-ethyl-3-methylimidazolium trifluoromethanesulfonate, 1-ethyl-3-methylimidazolium acetate, 1-ethyl-3-methylimidazolium methanesulfonate, 1-ethyl-3-methylimidazolium chloride, 1-ethyl-3-methylimidazolium sulfate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, 1-ethyl-3-methylimidazolium tetrafluoroborate, choline trifluoromethanesulfonate, choline saccharinate, choline acesulfamate, choline acetate, and mixtures thereof.
36. Conductive pressure sensitive adhesive composition according to statement 35, characterized in that the ionic liquid is present in an amount of 0.1 to 35 wt.-%, based on the total weight of the pressure sensitive adhesive composition.
37. Conductive pressure sensitive adhesive composition according to statement 35, characterized in that the ionic liquid is present in an amount of 0.5 to 25 wt.-%, based on the total weight of the pressure sensitive adhesive composition.
38. Conductive pressure sensitive adhesive composition according to statement 35, characterized in that the ionic liquid is present in an amount of 1 to 15 wt.-%, based on the total weight of the pressure sensitive adhesive composition.
39. Conductive pressure sensitive adhesive composition according to any of the forgoing statements, characterized in that the composition comprises a copolymer obtained from a reaction mixture comprising:
a) 2-EHA in an amount of 1 to 20 wt.-%, preferably 2 to 7 wt.-%
b) MMA in an amount of 1 to 7 wt.-%, preferably 2 to 5 wt.-%
c) 2-HEA in an amount of 15 to 40 wt.-%, preferably 20 to 35 wt.-%
d) BA in an amount of 40 to 80 wt.-%, preferably 55 to 65 wt.-%
e) compound of Formula (I) in an amount of 0.1 to 50 wt.-%, preferably 0.2 to 30 wt.-%, especially 0.5 to 10 wt.-%,
based on the total weight of the reaction mixture; and
an ionic liquid in of 0.1 to 35 wt.-%, preferably 0.5 to 25 wt.-%, especially 1 to 15 wt.-%, based on the total weight of the pressure sensitive adhesive composition.
   40. Process for the production of a conductive pressure sensitive adhesive composition according to any of statements 1 to 39, the process comprising the following steps:
a) providing an initial and a feed solution of a reaction mixture comprising a monomeric acrylic mixture and a compound of formula (I);
b) providing an initial and a feed solution of an initiator solution;
c) adding the initial initiator solution to the initial solution of the reaction mixture at an ambient temperature;
d) heating the reaction mixture of step c) to temperatures between 60° C. and 120° C., preferably to temperatures between 70° C. and 90° C.;
e) continuously adding the feed solution of the reaction mixture and feed solution of the initiator solution.
   41. Process according to statement 40, characterized in that the initiator is selected from the group consisting of 2,2′-azobisisobutyronitril, 1,1′-Azobis(cyclohexanecarbonitrile), Dimethyl 2,2′-azobis(2-methylpropionate), 2,2′-Azobis(2,4-dimethylvaleronitrile), 2,2′-Azobis(2-methylbutyronitrile), Benzoyl peroxide, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide.
   42. Use of a pressure sensitive adhesive composition according to any of statements 1 to 39 in skin applications as a contact medium as part of electrodes measuring bio-signals from the skin, athletic tape, transdermal drug patches, permanent adhesive tapes for industrial use and permanent adhesive tapes for construction work and/or for safety labels for power equipment, masking tapes, price marking labels and/or electrochemical debonding.
   43. A compound of Formula (I):

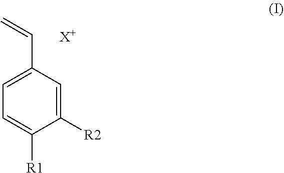

wherein R¹ is either a sulfate group (OSO₃⁻), a sulfonate group (SO₃⁻), or a hydrogen atom (H); R² is a sulfate group, a sulfonate group, an alkoxy group or a hydrogen atom, and wherein X⁺ is a counter ion with the proviso that either R¹ or R² is a sulfate group.
44. A compound according to statement 43, characterized in that R1 is a sulfate group (OSO₃⁻).
45. A compound according to statement 44, characterized in that R2 is a hydrogen atom.
46. A compound according to statement 44, characterized in that R2 is an alkoxy group.
47. A compound according to statement 43, characterized in that R2 is a sulfate group (OSO₃⁻).
48. A compound according to statement 47, characterized in that R1 is a hydrogen atom.
49. A compound according to statement 47, characterized in that R1 is an alkoxy group.
50. A compound according to any of the statements 43 to 49, characterized in that X⁺ is selected from the group of NR³R⁴R⁵R⁶⁺, with each of R³, R⁴, R⁵ and R⁶ being independently selected from C₁-C₈-alkyl, preferably C₂-C₆-alkyl, more preferably X⁺ is N(C₄H₉)₄_{⁺}.
51. A compound according to any of the statements 43 to 50, characterized in that X⁺ is an optionally substituted imidazolium.
52. A compound according to statement 51, characterized in that X⁺ is imidazolium.
53. A compound according to statement 51, characterized in that X⁺ is a substituted imidazolium, preferably a 1,3-dialkylimidazolium, with each alkyl group being independently selected from a linear or branched C1-C6 alkyl, more preferably a linear or branched C1-C3 alkyl.
54. A compound according to statement 53, characterized in that X⁺ is a 1,3-dialkylimidazolium, with each alkyl group being independently selected from a linear C1-C6 alkyl, preferably a linear C1-C3 alkyl.
55. A compound according to statement 53, characterized in that X⁺ is 1-ethyl-3-methylimidazolium.
56. A compound according to any of the statements 43 to 55, characterized in that X⁺ is choline.
57. A compound according to any of the statements 43 to 56, characterized in that X⁺ is an optionally substituted pyridinium.
58. A compound according to statement 57, characterized in that X⁺ is pyridinium.
59. A compound according to statement 57, characterized in that X⁺ is a substituted pyridinium, preferably a singly-N-substituted pyridinium or a doubly-substituted pyridinium, i.e. with substituents at the 1,2 or 1,3, or 1,4 positions on the pyridine ring, more preferably X⁺ is a 1-alkylpyridinium, a 1,2-alkylpyridinium, 1,3-alkylpyridinium or a 1,4-alkylpyridinium, with each alkyl group being independently selected from a linear or branched C1-C6 alkyl, preferably a linear or branched C1-C4 alkyl, more preferably a linear C1-C4 alkyl.
60. A compound according to any of the statements 43 to 59, characterized in that X⁺ is an optionally substituted pyrrolidinium.
61. A compound according to statement 60, characterized in that X⁺ is pyrrolidinium.
62. A compound according to statement 60, characterized in that X⁺ is a substituted pyrrolidinium, preferably a singly-N-substituted pyrrolidinium or a doubly-substituted pyrrolidinium, for example, the nitrogen ring member being doubly-substituted, more preferably a 1-alkylpyrrolidinium or a 1,1-dialkyl pyrrolidinium, with each alkyl group being independently selected from a linear or branched C1-C6 alkyl, preferably a linear or branched C1-C4 alkyl, more preferably a linear C1-C4 alkyl.
63. A compound according to any of the statements 43 to 62, characterized in that X⁺ is phosphonium.
64. A compound according to any one of statements any of the statements 43 to 63, characterized in that X⁺ is sulfonium.
65. A compound according to statement 43, which has the formula (IA)

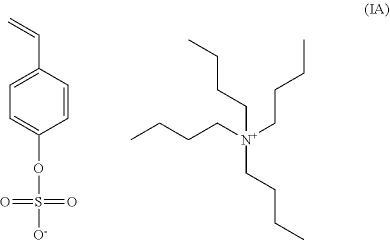 [SMILES: C=Cc1ccc(OS(=O)(=O)[O-])cc1 ; CCCC[N+](CCCC)(CCCC)CCCC]

66. Use of a compound as defined in any of the statements 43 to 65 for use as a monomer in a polymerization process.

The present invention will be described in more detail with reference to the examples below. However, these are in no way to be understood as limiting the scope and spirit of the invention.

### EXAMPLE 1

i) Preparation of the Following Monomer of the Formula (IA), which is Abbreviated in the Following by SS (Styrene Sulfate):

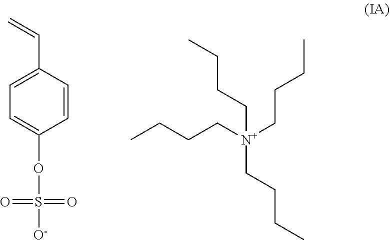 [SMILES: C=Cc1ccc(OS(=O)(=O)[O-])cc1 ; CCCC[N+](CCCC)(CCCC)CCCC]

The reaction was carried out in a three-necked flask with a reflux condenser and a magnetic stirrer. Under nitrogen, 4-Vinylphenol (4-VP, 6.0 g, 50.0 mmol) and sulfur trioxide trimethylamine complex (SO3NMe, 10.5 g, 75.0 mmol, 1.5 eq) were added to the flask and dissolved in 60 mL of anhydrous DMF. The reaction was stirred at T=80° C. oil bath temperature for 72 hours.

The reaction was cooled down to room temperature, and 1.1 eq of tetrabutylammonium hydroxide (TBA OH, 40% aqueous solution) was added and stirred for one hour. More water was added, and the solution was extracted with DCM. The solvent was evaporated, and the product was purified by column chromatography in 95:5 DCM: MeOH. The product was dried in vacuo and solidified to an off-white solid. Isolated yield of SS: 60%.

### EXAMPLE 2

### i) Preparation of Conductive Pressure Sensitive Adhesive (PSA) Compositions

The compositions of table 1 were prepared in the following manner:

An initiator solution was added to an initial monomeric acrylic mixture containing of 2-EHA, MMA, 2-HEA, BA and the compound of formula (IA) as depicted above and heated to 80° C. while stirring. Additional solution of monomeric acrylic mixture and initiator solution were continuously added. The mixture was then cooled to room temperature and diluted with ethanol, isopropanol and ethyl acetate to obtain the final polymer solution.

TABLE 1

| | acrylic mixture | |
|---|---|---|
| | (2-EHA, MMA, 2- | SS |
| Batch | HEA, BA) [wt.-%] | [wt.-%] |
| A* | 100 | 0 |
| B | 99.5 | 0.5 |
| C | 98.5 | 1.5 |
| D | 95 | 5 |

*comparative

### ii) Preparation of PSA Films and Procedure for Impedance Measurements

The PSA films for property testing were prepared by casting a film of the respective polymer solution on silicone paper. After application, the film was dried in an oven at 115° C. for 5 min and covered with silicone paper. Film thickness was adjusted to obtain films with a coating weight ρ_{A} of 30 g/m². For each polymer solution, two 2×2 cm testing strips were applied. The two substrates were then glued together at an angle of 180° and impedance measurements at a frequency range 9*106 Hz≤v≤0.1 Hz were performed with a potentiostat. The resistivity ρ was calculated as shown in equation 1 with the measured impedance Z, overlap area A and layer thickness d.

$$ρ = \frac{Z \cdot A}{d} \qquad ( 1 )$$

iii) Decrease in Resistivity by the Compound of Formular (I)

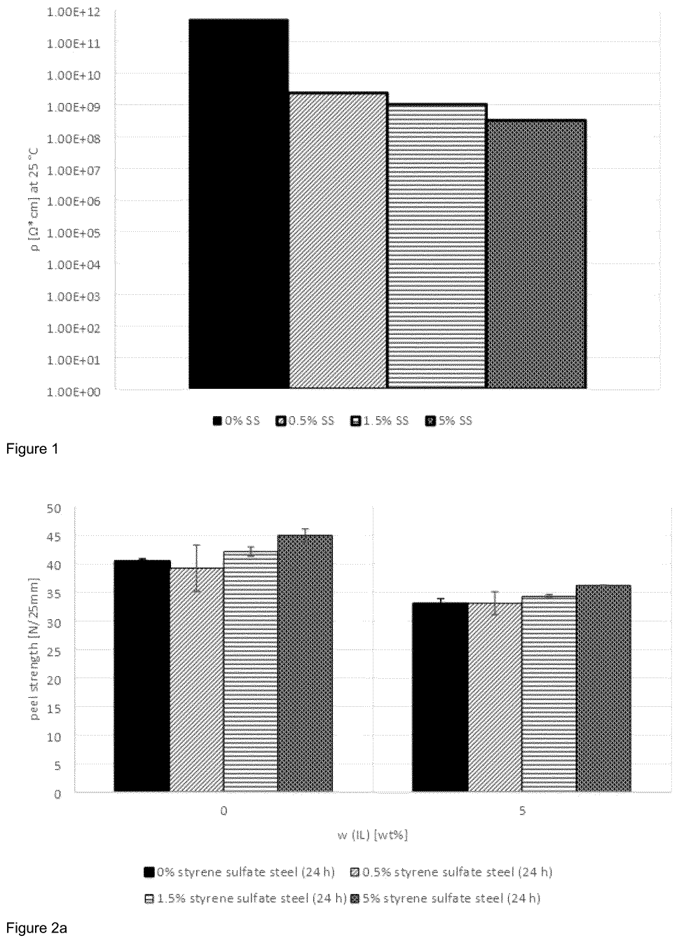

Data gathered in impedance measurements is listed in Table 2 and visualized in FIG. 1. The resistivity of the PSA decreases with increasing content of the compound of Formular (I). As conductivity is defined as the reciprocal resistivity, the conductivity increases with increasing content of the compound of Formula (I). This can be an advantage for applications in the electronics industry or the medical industry, such as ECG medical patches. As is apparent from the data of FIG. 1, resistivity decreases with increasing SS content.

TABLE 2

| w (SS) | ρ |
|---|---|
| wt %. | 10⁸ Ω*cm |
| 0 | 4825 |
| 0.5 | 22.74 |
| 1.5 | 9.984 |
| 5 | 3.101 |

### EXAMPLE 3

### i) Preparation of Polymer-Ionic Liquid Blends

Polymer solutions were prepared as described in example 2. The ionic liquid (IL) 1-Ethyl-3-methylimidazolium triflate was added to prepare polymer-ionic liquid blends. Blends with 5 wt % or 10 wt % of the ionic liquid with regard to the solid content of the polymer solution were prepared.

### ii) Preparation of PSA Films for Peel Tests

The PSA films for property testing were prepared by applying the respective polymer solution on an etched polyethylene foil. After application, the film was dried in an oven at 115° C. for 5 min and covered with silicone paper. Film thickness was adjusted to obtain films with a coating weight ρ_{A} of 30 g/m².

iii) Preparation of 180° Peel Test Samples and Procedure

180° peel tests were performed according to protocol FTM1 defined by European association for the self-adhesive label industry (FINAT).

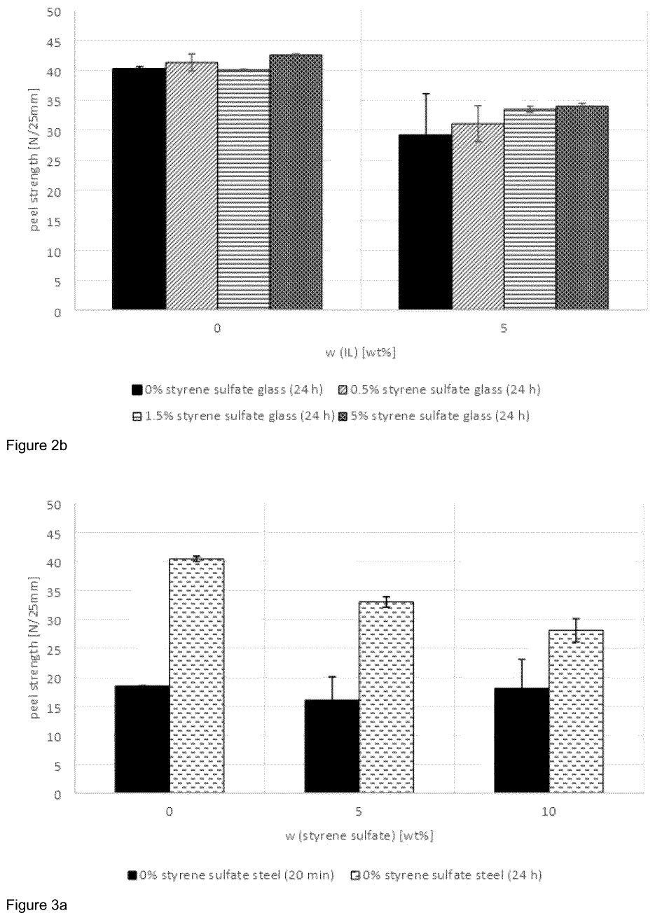

Data gathered in 180° peel tests of the polymer-ionic liquid blends is listed in Table 3 and visualized in FIG. 2. Without IL and with 5 wt % IL, the presence of a compound of Formula (I) in the polymer increases the adhesion of the PSA on stainless steel and glass substrates. Peel strength increases with increasing content of a compound of Formula (I). An increase is desirable for high performance PSAs. FIG. 2 summarizes the evaluation of 180° peel tests 24 hours after application on stainless steel (FIG. 2a) and glass (FIG. 2b). Tests were performed with crude polymer solutions (left) and polymer-ionic liquid blends with 5 wt % IL (right) with 0 (solid), 0.5 (diagonal), 1.5 (horizontal) and 5 wt % of a compound of Formula (I) (crossed). As is apparent from the data provided, peel strength increases with increasing content of a compound of Formula (I) on both substrates with and without IL added.

TABLE 3

| | w (SS) | w (IL) | Peel strength |
|---|---|---|---|
| Substrate | wt %. | wt %. | [N/25 mm] |
| Stainless steel | 0 | 0 | 40.3 ± 0.5 |
| Stainless steel | 0.5 | 0 | 39 ± 4 |
| Stainless steel | 1.5 | 0 | 42.1 ± 0.8 |
| Stainless steel | 5 | 0 | 45 ± 2 |
| Stainless steel | 0 | 5 | 32.9 ± 0.9 |
| Stainless steel | 0.5 | 5 | 33 ± 2 |
| Stainless steel | 1.5 | 5 | 34.2 ± 0.3 |
| Stainless steel | 5 | 5 | 36.13 ± 0.02 |
| Glass | 0 | 0 | 40.1 ± 0.6 |
| Glass | 0.5 | 0 | 41 ± 2 |
| Glass | 1.5 | 0 | 40.0 ± 0.2 |
| Glass | 5 | 0 | 42.5 ± 0.2 |
| Glass | 0 | 5 | 29 ± 7 |
| Glass | 0.5 | 5 | 31 ± 3 |
| Glass | 1.5 | 5 | 33.4 ± 0.5 |
| Glass | 5 | 5 | 33.9 ± 0.5 |

### iv) Synergistic Effect of SS and IL on Substrate Wetting

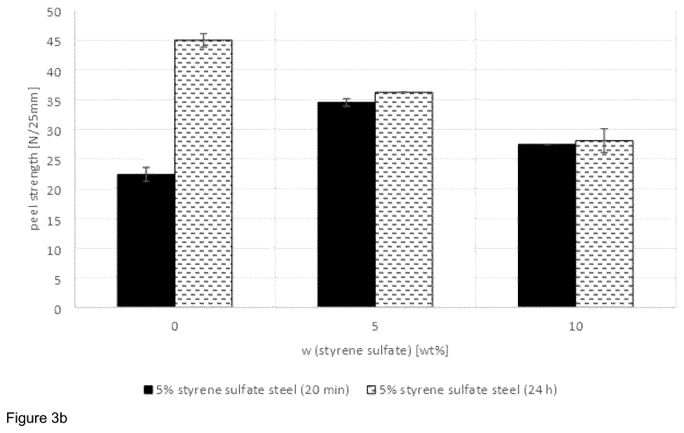

Data gathered in 180° peel tests of polymer-ionic liquid blends is listed in Table 4 and visualized in FIG. 3. If no comonomers of Formula (I) are present in the polymer, the adhesive needs time to reach its maximum strength independent from the addition of ionic liquid as shown by the significant difference in peel strength measured 20 minutes and 24 hours after application. The same observation is made for polymers containing 5 wt % of the compound of Formula (I), but no ionic liquid. However, if IL is added to a polymer with comonomers of Formula (I), peel strength 20 minutes after application increases significantly compared to the previously mentioned systems. Maximum peel strength is almost reached after 20 minutes. This observation shows a synergistic effect between the presence of comonomers of Formula (I) in the polymer and ILs which positively affects the wetting process in terms of a shortened application time.

With reference to FIG. 3, evaluation data of 180° peel tests of polymer-ionic liquid blends on stainless steel are shown. Tests were performed 20 minutes (solid) and 24 hours (dotted) after application with 0, 5 and 10 wt % IL and 0 wt % of a compound of Formula (I) (FIG. 3a) and 10 wt % of a compound of Formula (I) (FIG. 3b). Without comonomers of Formula (I) and without IL, a significant difference in peel strength is observed over time. With comonomers of Formula (I) and IL, maximum peel strength is achieved after 20 minutes which shows the synergistic effect of the presence of comonomers of Formula (I) in the polymer and ILs on the wetting of the substrate by the polymer.

TABLE 4

| | | | Time after | |
|---|---|---|---|---|
| | w (SS) | w (IL) | application | Peel strength |
| Substrate | wt %. | wt %. | [h] | [N/25 mm] |
| Stainless steel | 0 | 0 | 0.33 | 17.42 ± 0.05 |
| Stainless steel | 0 | 0 | 24 | 40.3 ± 0.5 |
| Stainless steel | 0 | 5 | 0.33 | 16 ± 4 |
| Stainless steel | 0 | 5 | 24 | 32.9 ± 0.9 |
| Stainless steel | 0 | 10 | 0.33 | 18 ± 5 |
| Stainless steel | 0 | 10 | 24 | 28 ± 2 |
| Stainless steel | 5 | 0 | 0.33 | 22 ± 2 |
| Stainless steel | 5 | 0 | 24 | 45 ± 2 |
| Stainless steel | 5 | 5 | 0.33 | 34.4 ± 0.7 |
| Stainless steel | 5 | 5 | 24 | 36.13 ± 0.02 |
| Stainless steel | 5 | 10 | 0.33 | 27.4 ± 0.3 |
| Stainless steel | 5 | 10 | 24 | 28 ± 2 |

The data provided not only shows the improved adhesion properties of the conductive pressure sensitive adhesive composition of the present invention, but also evidences the synergistic effects which can be achieved by combination with an ionic liquid.

1: A conductive pressure sensitive adhesive composition comprising a copolymer obtained from a reaction mixture comprising:
a) a monomeric acrylic mixture; and
b) a compound of Formula (I): 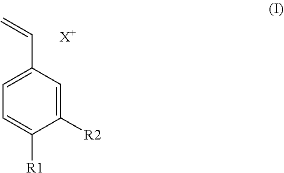
wherein R¹ is either a sulfate group (OSO₃⁻), a sulfonate group (SO₃⁻), or a hydrogen atom (H);
wherein R² is a sulfate group, a sulfonate group, an alkoxy group or a hydrogen atom
and wherein X⁺ is a counter ion when either R¹ or R² is a sulfate group.

2: The conductive pressure sensitive adhesive composition according to claim 1, characterized in that R1 is a sulfate group (OSO₃⁻).

3: The conductive pressure sensitive adhesive composition according to claim 2, characterized in that R2 is a hydrogen atom.

4: The conductive pressure sensitive adhesive composition according to claim 1, characterized in that R2 is a sulfate group (OSO₃⁻).

5: The conductive pressure sensitive adhesive composition according to claim 1, characterized in that X⁺ is selected from the group consisting of NR³R⁴R⁵R⁶⁺, with each of R³, R⁴, R⁵ and R⁶ independently selected from C₂-C₆-alkyl, 1-ethyl-3-methylimidazolium, choline, substituted pyrrolidinium, substituted phosphonium, substituted sulfonium, and mixtures thereof.

6: (canceled)

7: The conductive pressure sensitive adhesive composition according to claim 1, characterized in that X⁺ is N(C₄H₉)₄_{⁺}.

8: The conductive pressure sensitive adhesive composition according to claim 1, characterized in that the monomeric acrylic mixture comprises at least one of the following: methyl methacrylate, hydroxyethyl acrylate, hydroxypropyl acrylate, hydroxybutyl acrylate, n-butyl acrylate, 2-ethylhexyl acrylate, acrylic acid, C1-C18 alkyl (meth)acrylate, (meth)acrylamide, vinyl acetate, N-vinyl caprolactame, acrylonitrile, vinyl ether, benzyl (meth)acrylate, cyclohexyl (meth)acrylate, glycidyl (meth)acrylate or mixtures thereof.

9: The conductive pressure sensitive adhesive composition according to claim 1, characterized in that the compound of Formula (I) is contained in an amount of 0.5 to 10 wt.-%, based on the total weight of the reaction mixture.

10: The conductive pressure sensitive adhesive composition according to claim 8, characterized in that the monomeric acrylic mixture contains 2-ethylhexyl acrylate (2-EHA) in an amount of 2 to 7 wt.-%, based on the total weight of the reaction mixture.

11: The conductive pressure sensitive adhesive composition according to claim 8, characterized in that the monomeric acrylic mixture contains methyl methacrylate (MMA) in an amount of 2 to 5 wt.-%, based on the total weight of the reaction mixture.

12: The conductive pressure sensitive adhesive composition according to claim 8, characterized in that the monomeric acrylic mixture contains hydroxyethyl acrylate (2-HEA) in an amount of 20 to 35 wt.-%, based on the total weight of the reaction mixture.

13: The conductive pressure sensitive adhesive composition according to claim 1, characterized in that the monomeric acrylic mixture contains butyl acrylate (BA) in an amount of 55 to 65 wt.-%, based on the total weight of the reaction mixture.

14: The conductive pressure sensitive adhesive composition according to claim 1 further comprises an ionic liquid, selected from the group consisting of 1-ethyl-3-methylimidazolium trifluoromethanesulfonate, 1-ethyl-3-methylimidazolium acetate, 1-ethyl-3-methylimidazolium methanesulfonate, 1-ethyl-3-methylimidazolium chloride, 1-ethyl-3-methylimidazolium sulfate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, 1-ethyl-3-methylimidazolium tetrafluoroborate, choline trifluoromethanesulfonate, choline saccharinate, choline acesulfamate, choline acetate, and mixtures thereof.

15: The conductive pressure sensitive adhesive composition according to claim 14, characterized in that the ionic liquid is present in an amount of 1 to 15 wt.-%, based on the total weight of the pressure sensitive adhesive composition.

16: The conductive pressure sensitive adhesive composition according to claim 1, characterized in that the composition comprises a copolymer obtained from a reaction mixture comprising:
a) 2-EHA in an amount of 2 to 7 wt.-%,
MMA in an amount of 2 to 5 wt.-%,
2-HEA in an amount of 20 to 35 wt.-%,
BA in an amount of 55 to 65 wt.-%; and
b) compound of Formula (I) in an amount of 0.5 to 10 wt.-%,
c) an ionic liquid in an amount of 1 to 15 wt.-%,
wherein the wt % of components a) and b) are based on the total weight of the reaction mixture and the wt % of component c) is based on the pressure sensitive adhesive composition.

17: A process for preparing the conductive pressure sensitive adhesive composition, the process comprising the following steps:
a) providing an initial and a feed solution of a reaction mixture comprising:
a monomeric acrylic mixture and a compound of Formula (I); 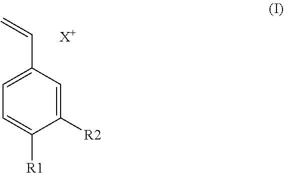
b) providing an initial and a feed solution of an initiator solution;
c) adding the initial initiator solution to the initial solution of the reaction mixture at an ambient temperature;
d) heating the reaction mixture of step c) to temperatures between 70° C. and 90° C.; and
e) continuously adding the feed solution of the reaction mixture and feed solution of the initiator solution.

18: The process according to claim 17, characterized in that the initiator is selected from the group consisting of 2,2′-azobisisobutyronitril, 1,1′-Azobis(cyclohexanecarbonitrile), Dimethyl 2,2′-azobis(2-methylpropionate), 2,2′-Azobis(2,4-dimethylvaleronitrile), 2,2′-Azobis(2-methylbutyronitrile), Benzoyl peroxide, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, and lauroyl peroxide.

19: An article comprising the conductive pressure sensitive adhesive composition according to claim 1 which is a skin tape, athletic tape, transdermal drug patch, permanent adhesive tape, safety label, masking tape, price marking label, electrochemical debonding tape, medical applicator, or diagnostic applicator.

20: (canceled)

21: The conductive pressure sensitive adhesive composition of claim 1 wherein the Formula (I) is (IA):

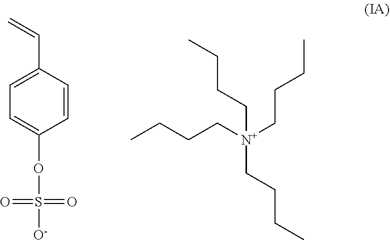 [SMILES: C=Cc1ccc(OS(=O)(=O)[O-])cc1 ; CCCC[N+](CCCC)(CCCC)CCCC]

22: (canceled)

23: The conductive pressure sensitive adhesive composition according to claim 8, wherein the monomeric acrylic mixture is selected from the group consisting of hydroxyethyl acrylate, methyl methacrylate, n-butyl acrylate, 2-ethylhexyl acrylate and mixtures thereof.
